(19) United States
(12) Patent Application Publication
(10) Pub. No.: US 2014/0170135 A1
(43) Pub. Date: Jun. 19, 2014

(54) EGFR EXPRESSION IS ASSOCIATED WITH DECREASED BENEFIT FROM TRASTUZUMAB IN THE NCCTG N9831 TRIAL

(71) Applicants: David L. RIMM, Branford, CT (US); Huan CHENG, Jiangsu (CN); Anastasios DIMOU, Philadelphia, PA (US); Valsamo ANAGNOSTOU, New Haven, CT (US)

(72) Inventors: David L. RIMM, Branford, CT (US); Huan CHENG, Jiangsu (CN); Anastasios DIMOU, Philadelphia, PA (US); Valsamo ANAGNOSTOU, New Haven, CT (US)

(73) Assignee: YALE UNIVERSITY, New Haven, CT (US)

(21) Appl. No.: 14/098,257

(22) Filed: Dec. 5, 2013

Related U.S. Application Data
(60) Provisional application No. 61/733,849, filed on Dec. 5, 2012.

(51) Int. Cl. G01N 33/574; A61K 31/337; A61K 31/704; A61K 31/675; A61K 39/395; A61K 31/517

(52) U.S. Cl. CPC G01N 33/574; A61K 39/39558; A61K 31/517; A61K 31/704; A61K 31/675; A61K 31/337
USPC 424/133.1; 514/266.24; 435/7.23

(57) ABSTRACT

The subject invention provides a method for treating a patient having a tumor associated with HER2+ breast cancer comprising: measuring in a sample of the patient's tumor a level of expression of epidermal growth factor receptor protein by using an antibody that detects the cytoplasmic domain of epidermal growth factor receptor; and comparing the level of expression of epidermal growth factor receptor protein in the patient's tumor so measured with a reference value; wherein if the level of expression of epidermal growth factor receptor measured is higher than the reference value, treating the patient with a therapy other than trastuzumab.

This application claims benefit of U.S. Provisional Application No. 61/733,849, filed Dec. 5, 2012, the entire content of which is hereby incorporated by reference herein.

This invention was made with government support under NIH CA-139431, CA-129949, and CA025224 awarded by National Institute of Health. The government has certain rights in the invention.

### FIELD OF THE INVENTION

This invention relates to the field of treating patients having tumors associated with HER2+ breast cancer by measuring the level of epidermal growth factor receptor protein expression to predict the patient's response to trastuzumab.

### BACKGROUND OF THE INVENTION

Trastuzumab is a humanized monoclonal antibody against the extracellular domain of human epidermal growth factor receptor 2 (HER2), which has shown therapeutic benefit in the 15%-20% of breast cancer cases where this molecule is over-expressed. When added to chemotherapy in the adjuvant setting, disease free survival at 4 years has been demonstrated to be 85.7%, compared to 73.7% in the control arm (Perez et al., J Clin Oncol 29:3366-73, 2011).

Despite its significant improvement of outcome in HER2 positive disease, not all patients benefit from trastuzumab. In the metastatic setting, around 50% of the patients do not achieve more than 50% tumor shrinkage representing de novo drug resistance even when it is combined with chemotherapy (Zito et al., PLoS One 7:e31331, 2012). As the number of therapeutic options increases for those with HER2 over-expressing breast cancer, patients would benefit from new biomarkers to increase the specificity of companion diagnostic tests to better predict benefit for specific anti-HER2 therapies.

### SUMMARY OF THE INVENTION

The present invention is based on a method for selecting treatment for HER2+ breast cancer patients. Particularly it was found that by measuring in a sample of the patient's tumor the level of expression of epidermal growth factor receptor protein by using an antibody that detects the cytoplasmic domain of epidermal growth factor receptor; and comparing the level of expression of epidermal growth factor receptor protein in the patient's tumor so measured with a reference value; a particular treatment which would be more beneficial to the patient could be chosen. For example, patients who are measured to have a level of expression of epidermal growth factor receptor which is higher than a reference value should be selected for a treatment other than chemotherapy involving concurrent administration of trastuzumab.

The subject invention provides a method for treating a patient having a tumor associated with HER2+ breast cancer comprising a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; wherein if the level of expression of epidermal growth factor receptor measured in step a) is higher than the reference value, treating the patient with a therapy other than trastuzumab.

The subject invention also provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; wherein if the level of expression of epidermal growth factor receptor measured in step a) is higher than the reference value, treating the patient with a therapy other than chemotherapy involving concurrent administration of trastuzumab.

The subject invention also provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein in the patient's tumor so measured with a reference value; and c) treating the patient with a therapy other than chemotherapy involving concurrent administration of trastuzumab if the level of expression of epidermal growth factor receptor measured in step a) is higher than the reference value.

The subject invention also provides a method for selecting a therapeutic treatment or combination of therapeutic treatments for a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; selecting a therapeutic treatment or combination of therapeutic treatments based upon the results of the comparison in step b).

The subject invention also provides a method for determining the likelihood that a patient having a tumor associated with breast cancer will benefit from trastuzumab administered concurrently with a chemotherapy comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein in the patient's tumor so measured with a reference value of epidermal growth factor receptor protein expression associated with disease specific survival in patients having tumors associated with breast cancer who have received trastuzumab administered concurrently with the chemotherapy; so as to thereby determine the likelihood that the patient will benefit from trastuzumab administered concurrently with the chemotherapy.

The subject invention also provides a method for determining the likelihood that a patient having a tumor associated with breast cancer will have an increased probability of disease specific survival from receiving trastuzumab administered concurrently with a chemotherapy comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein so measured with a reference value of epidermal growth factor receptor protein expression associated with disease specific survival in patients having tumors associated with breast cancer who have received trastuzumab administered concurrently with the chemotherapy; so as to thereby determine the likelihood that the patient will have an increased probability of disease specific survival.

The subjection invention also provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein so measured with a reference value; so as to thereby treat the patient by determining whether the patient will benefit from treatment comprising a chemotherapy and concurrent administration of trastuzumab.

The subject invention also provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; wherein if the level of expression of epidermal growth factor receptor measured in step a) is higher than the reference value, treating the patient with an EGFR inhibitor therapy and trastuzumab.

The subject invention also provides a method for determining the likelihood that a patient having a tumor associated with, breast cancer will benefit from trastuzumab comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein in the patient's tumor so measured with a reference value; so as to thereby determine the likelihood that the patient will benefit from trastuzumab.

The subject invention also provides a method for determining the likelihood that a patient having a tumor associated with breast cancer will have an increased probability of disease specific survival from receiving trastuzumab comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein so measured with a reference value; so as to thereby determine the likelihood that the patient will have an increased probability of disease specific survival.

The subject invention also provides a method for determining the likelihood that a HER2+ breast cancer patient will benefit from trastuzumab comprising measuring the level of expression of epidermal growth factor receptor in a tumor specimen obtained from the patient comprising: a) staining a tissue sample of the tumor with a first stain that labels a subcellular compartment wherein the subcellular compartment is a non-nuclear compartment and a second stain that labels a biomarker, wherein the biomarker is epidermal growth factor receptor protein and wherein the second stain is specific for the cytoplasmic domain of epidermal growth factor receptor; b) obtaining a high resolution digital image of each of the epidermal growth factor receptor and of the subcellular compartment; c) analyzing the digital images to determine a quantitative level of expression of epidermal growth factor receptor protein present in the subcellular compartment; and d) comparing the level of expression of epidermal growth factor receptor protein so determined with a reference value; thereby determining the level of expression of epidermal growth factor receptor protein in the tumor, wherein if the level of expression of epidermal growth factor receptor is higher than the reference value, it is unlikely the patient will benefit from trastuzumab.

### BRIEF DESCRIPTION OF THE FIGURES

FIG. 1: a consort diagram describing the patients registered to the N9831 trial and the patients in each arm of the trial.

FIG. 2: a DFS comparison of EGFR high group versus EGFR not high group in the N9831 cohort, stratified by treatment arm. FIG. 2A: Arm A patients; Kaplan-Meier curves for low EGFR patients and high EGFR patients. FIG. 2B: Arm B patients; Kaplan-Meier curves for low EGFR patients and high EGFR patients. FIG. 2C: Arm C patients; Kaplan-Meier curves for low EGFR patients and high EGFR patients.

FIG. 3: survival curves for each trial arm in N9831 patients with low (A) or high (B) EGFR levels.

FIG. 4 illustrates that patients with high level of EGFR (defined by the cut-point from the N9831 cohort) show less benefit from trastuzumab treatment in metastatic breast cancer cohort.

### DETAILED DESCRIPTION OF THE INVENTION

Epidermal Growth Factor Receptor (EGFR, HER1) is known to heterodimerize with HER2 and has been hypothesized to modulate the effectiveness of anti-HER2 therapy such as trastuzumab. Testing this hypothesis has been historically challenging due to difficulties in the tissue testing for this marker. The present invention is based on use of a new, standardized, quantitative immunofluorescence assay and a novel EGFR antibody to evaluate the correlation between EGFR expression and clinical outcome. Surprisingly it was found that high expression of EGFR is associated with decreased benefit from adjuvant concurrent (not sequential) trastuzumab and shorter PFS in the metastatic setting.

Accordingly, the subject invention provides a method for treating a patient having a tumor associated with HER2+ breast cancer comprising a) measuring in a sample of the patient's tumor (e.g., a metatstatic breast cancer tumor sample) a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; wherein if the level of expression of epidermal growth factor receptor measured in step a) is higher than the reference value, treating the patient with a therapy other than trastuzumab.

In one embodiment, if the level of expression of epidermal growth factor receptor measured in step b) is higher than the reference value, treating the patient with a therapy other than chemotherapy involving concurrent administration of trastuzumab.

In one embodiment, the level of expression is determined using a quantitative image analysis procedure.

In one embodiment, the level of expression is determined using an automated pathology system.

In one embodiment, the reference value is thirteen nanograms of epidermal growth factor receptor protein per microgram of total protein.

In one embodiment, the antibody detects the epidermal growth factor receptor epitope that is bound by D38B1.

In one embodiment, the antibody has the specificity of D38B1.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In another embodiment, the tissue is formalin-fixed paraffin-embedded tissue.

In one embodiment, the reference value comprises the median level of expression of epidermal growth factor receptor protein from a patient population of HER2+ breast cancer patients.

The subject invention provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; wherein if the level of expression of epidermal growth factor receptor measured in step a) is higher than the reference value, treating the patient with a therapy other than chemotherapy involving concurrent administration of trastuzumab.

In one embodiment, the level of expression is determined using a quantitative image analysis procedure.

In one embodiment, the level of expression is determined using an automated pathology system.

In one embodiment, the reference value is thirteen nanograms of epidermal growth factor receptor protein per microgram of total protein.

In one embodiment, the antibody detects the epidermal growth factor receptor epitope that is bound by D38B1.

In one embodiment, the antibody has the specificity of D38B1.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In another embodiment, the tissue is formalin-fixed paraffin-embedded tissue.

In one embodiment, the reference value comprises the median level of expression of epidermal growth factor receptor protein from a patient population of HER2+ breast cancer patients.

The subject invention also provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein in the patient's tumor so measured with a reference value; and c) treating the patient with a therapy other than chemotherapy involving concurrent administration of trastuzumab if the level of expression of epidermal growth factor receptor measured in step a) is higher than the reference value.

In one embodiment, the level of expression is determined using a quantitative image analysis procedure.

In one embodiment, the level of expression is determined using an automated pathology system.

In one embodiment, the reference value is thirteen nanograms of epidermal growth factor receptor protein per microgram of total protein.

In one embodiment, the antibody detects the epidermal growth factor receptor epitope that is bound by D38131.

In one embodiment, the antibody has the specificity of D3831.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In another embodiment, the tissue is formalin-fixed paraffin-embedded tissue.

In one embodiment, the reference value comprises the median level of expression of epidermal growth factor receptor protein from a patient population of HER2+ breast cancer patients.

The subject invention provides a method for selecting a therapeutic treatment or combination of therapeutic treatments for a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; selecting a therapeutic treatment or combination of therapeutic treatments based upon the results of the comparison in step b).

In one embodiment, a therapy other than trastuzumab is selected when the level of expression of epidermal growth factor receptor measured in step a) is greater than the reference value.

In one embodiment, a therapy other than chemotherapy involving concurrent administration of trastuzumab is selected when the level of expression of epidermal growth factor receptor measured in step a) is greater than the reference value.

In one embodiment, the level of expression is determined using a quantitative image analysis procedure.

In one embodiment, the level of expression is determined using an automated pathology system.

In one embodiment, the reference value is thirteen nanograms of epidermal growth factor receptor protein per microgram of total protein.

In one embodiment, the antibody detects the epidermal growth factor receptor epitope that is bound by D38B1.

In one embodiment, the antibody has the specificity of D38B1.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

In one embodiment, the reference value comprises the median level of expression of epidermal growth factor receptor protein from a patient population of HER2+ breast cancer patients.

The subject invention provides a method for determining the likelihood that a patient having a tumor associated with breast cancer will benefit from trastuzumab administered concurrently with a chemotherapy comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein in the patient's tumor so measured with a reference value of epidermal growth factor receptor protein expression associated with disease specific survival in patients having tumors associated with breast cancer who have received trastuzumab administered concurrently with the chemotherapy; so as to thereby determine the likelihood that the patient will benefit from trastuzumab administered concurrently with the chemotherapy.

In one embodiment, the level of expression is determined using a quantitative image analysis procedure.

In one embodiment, the level of expression is determined using an automated pathology system.

In one embodiment, there is a greater likelihood that the patient will benefit from trastuzumab administered concurrently with the chemotherapy if the epidermal growth factor receptor level measured in step a) is lower than the reference value.

In one embodiment, the reference value is thirteen nanograms of epidermal growth factor receptor protein per microgram of total protein.

In one embodiment, the benefit from trastuzumab administered concurrently with the chemotherapy comprises disease-specific survival after five years.

In one embodiment, the antibody detects the epidermal growth factor receptor epitope that is bound by D38B1.

In one embodiment, the antibody has the specificity of D38B1.

In one embodiment, the measuring in step a) does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

The subject invention provides a method for determining the likelihood that a patient having a tumor associated with breast cancer will have an increased probability of disease specific survival from receiving trastuzumab administered concurrently with a chemotherapy comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein so measured with a reference value of epidermal growth factor receptor protein expression associated with disease specific survival in patients having tumors associated with breast cancer who have received trastuzumab administered concurrently with the chemotherapy; so as to thereby determine the likelihood that the patient will have an increased probability of disease specific survival.

In one embodiment, the level of expression is determined using a quantitative image analysis procedure.

In one embodiment, the level of expression is determined using an automated pathology system.

In one embodiment, the patient will have an increased probability of survival if the epidermal growth factor receptor level measured in step a) is lower than the reference value.

In one embodiment, the patient will have an increased probability of survival after five years.

In one embodiment, the reference value is thirteen nanograms of epidermal growth factor receptor protein per microgram of total protein.

In one embodiment, the antibody detects the epidermal growth factor receptor epitope that is bound by D38B1.

In one embodiment, the antibody has the specificity of D38B1.

In one embodiment, the level of expression of epidermal growth factor receptor protein is the level within a subcellular compartment.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

The subjection invention provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein so measured with a reference value; so as to thereby treat the patient by determining whether the patient will benefit from treatment comprising a chemotherapy and concurrent administration of trastuzumab.

In one embodiment, the patient will benefit from treatment comprising a chemotherapy and concurrent administration of trastuzumab if the epidermal growth factor receptor protein measure in step b) is greater than the reference value.

In one embodiment, the level of expression is determined using a quantitative image analysis procedure.

In one embodiment, the level of expression is determined using an automated pathology system.

In one embodiment, the reference value is thirteen nanograms of epidermal growth factor receptor protein per microgram of total protein.

In one embodiment, the antibody detects the epidermal growth factor receptor epitope that is bound by D3881.

In one embodiment, the antibody has the specificity of D38B1.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

In one embodiment, the reference value comprises the median level of expression of epidermal growth factor receptor protein from a patient population of HER2+ breast cancer patients.

The subjection invention provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein so measured with a reference value; so as to thereby treat the patient by determining whether the patient will benefit from treatment comprising trastuzumab.

The subject invention provides a method for treating a patient having a tumor associated with HER2+ breast cancer comprising: a) measuring in a sample of the patient's tumor a level of expression of epidermal growth factor receptor protein by using an antibody that detects the cytoplasmic domain of epidermal growth factor receptor; and b) comparing the level of expression of epidermal growth factor receptor protein in the patient's tumor so measured with a reference value; wherein if the level of expression of epidermal growth factor receptor measured in step b) is higher than the reference value, treating the patient with an EGFR inhibitor therapy and trastuzumab.

In one embodiment, if the level of expression of epidermal growth factor receptor measured in step a) is higher than the reference value, treating the patient with an EGFR inhibitor therapy and trastuzumab administered concurrently with chemotherapy.

In one embodiment, the level of expression is determined using a quantitative image analysis procedure.

In one embodiment, the level of expression is determined using an automated pathology system.

In one embodiment, the reference value is thirteen nanograms of epidermal growth factor receptor protein per microgram of total protein.

In one embodiment, the antibody detects the epidermal growth factor receptor epitope that is bound by D38B1.

In one embodiment, the antibody has the specificity of D38B1.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

In one embodiment, the reference value comprises the median level of expression of epidermal growth factor receptor protein from a patient population of HER2+ breast cancer patients.

The subject invention provides a method for determining the likelihood that a patient having a tumor associated with breast cancer will benefit from trastuzumab comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein in the patient's tumor so measured with a reference value; so as to thereby determine the likelihood that the patient will benefit from trastuzumab.

In one embodiment, the level of expression is determined using a quantitative image analysis procedure.

In one embodiment, the level of expression is determined using an automated pathology system.

In one embodiment, there is a greater likelihood that the patient will benefit from trastuzumab if the epidermal growth factor receptor level measured in step a) is lower than the reference value.

In one embodiment, there is a greater likelihood that the patient will benefit from trastuzumab administered concurrently with the chemotherapy if the epidermal growth factor receptor level measured in step a) is lower than the reference value.

In one embodiment, the reference value is thirteen nanograms of epidermal growth factor receptor protein per microgram of total protein.

In one embodiment, the benefit from trastuzumab administered concurrently with the chemotherapy comprises disease-specific survival after five years.

In one embodiment, the antibody detects the epidermal growth factor receptor epitope that is bound by D38B1.

In one embodiment, the antibody has the specificity of D38B1.

In one embodiment, the measuring in step a) does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

In one embodiment, the reference value comprises the median level of expression of epidermal growth factor receptor protein from a patient population of HER2+ breast cancer patients.

The subject invention provides a method for determining the likelihood that a patient having a tumor associated with breast cancer will have an increased probability of disease specific survival from receiving trastuzumab comprising: a) measuring in a sample of the patient's tumor a level of expression of epidermal growth factor receptor protein by using an antibody that detects the cytoplasmic domain of epidermal growth factor receptor; b) comparing the level of expression of epidermal growth factor receptor protein so measured with a reference value; so as to thereby determine the likelihood that the patient will have an increased probability of disease specific survival.

In one embodiment, the level of expression is determined using a quantitative image analysis procedure.

In one embodiment, the level of expression is determined using an automated pathology system.

In one embodiment, the patient will have an increased probability of survival if the epidermal growth factor receptor level measured in step a) is lower than the reference value.

In one embodiment, the patient will have an increased probability of survival after five years.

In one embodiment, the reference value is thirteen nanograms of epidermal growth factor receptor protein per microgram of total protein.

In one embodiment, the antibody detects the epidermal growth factor receptor epitope that is bound by D38B1.

In one embodiment, the antibody has the specificity of D38B1.

In one embodiment, the level of expression of epidermal growth factor receptor protein is the level within a subcellular compartment.

In one embodiment, the measuring in step a) using an antibody does not require protease digestion.

In one embodiment, the level of expression of epidermal growth factor receptor protein is the level within a subcellular compartment.

In one embodiment, the subcellular compartment is non-nuclear.

In one embodiment, the sample is a tissue sample.

In one embodiment, the tissue is formalin-fixed paraffin-embedded tissue.

In one embodiment, the reference value comprises the median level of expression of epidermal growth factor receptor protein from a patient population of HER2+ breast cancer patients.

The subject invention provides a method for determining the likelihood that a HER2+ breast cancer patient will benefit from trastuzumab comprising measuring the level of expression of epidermal growth factor receptor in a tumor specimen obtained from the patient comprising: a) staining a tissue sample of the tumor with a first stain that labels a subcellular compartment wherein the subcellular compartment is a non-nuclear compartment and a second stain that labels a biomarker, wherein the biomarker is epidermal growth factor receptor protein and wherein the second stain is specific for the cytoplasmic domain of epidermal growth factor receptor; b) obtaining a high resolution digital image of each of the epidermal growth factor receptor and of the subcellular compartment; c) analyzing the digital images to determine a quantitative level of expression of epidermal growth factor receptor protein present in the subcellular compartment; and d) comparing the level of expression of epidermal growth factor receptor protein so determined with a reference value; thereby determining the level of expression of epidermal growth factor receptor protein in the tumor, wherein if the level of expression of epidermal growth factor receptor is higher than the reference value, it is unlikely the patient will benefit from trastuzumab.

In one embodiment, if the level of expression of epidermal growth factor receptor is higher than the reference value, treating the patient with a therapy other than chemotherapy administered with concurrent trastuzumab.

In one embodiment, the tissue section is fixed.

In one embodiment, the antibody detects the epidermal growth factor receptor epitope that is bound by D38B1.

In one embodiment, the antibody has the specificity of D38B1.

In one embodiment, the reference value is thirteen nanograms of epidermal growth factor receptor protein per microgram of total protein.

In one embodiment, the reference value comprises the median level of expression of epidermal growth factor receptor protein from a patient population of HER2+ breast cancer patients.

In one embodiment of any of the above methods, a sample of the tumor is first obtained from a breast cancer patient.

For the foregoing embodiments, each embodiment disclosed herein is contemplated as being applicable to each of the other disclosed embodiments.

As used herein, the term “disease specific survival” may refer to the percentage of people in a study who have not died from a specific disease in a defined period of time. Only deaths from the disease are counted and patients who died from causes other than the disease are not counted.

As used herein, the term “reference value” may, for example, refer to the median level or the average level of expression of epidermal growth factor receptor protein in a patient population of HER2+ breast cancer patients.

The reference value may, for example, be 13 nanograms of epidermal growth factor receptor protein per microgram of total protein.

Therapies other than chemotherapy involving concurrent administration of trastuzumab that may be administered to a patient having a tumor associated with HER2+ breast cancer may include, for example, administration of an EGFR inhibitor or EGFR targeted therapy. Many other therapies for treating breast cancer are known in the art.

As used herein, the term “selecting” and “selected” in reference to a patient is used to mean that a particular patient is specifically chosen from a larger group of patients on the basis of (due to) the particular patient having a level of expression of epidermal growth factor receptor that is higher or lower than the reference value, e.g., the patient has a level of expression of epidermal growth factor receptor that is higher than the reference value. Similarly, “selectively treating” refers to providing treatment to a patient having a level of expression of epidermal growth factor receptor that is higher or lower than the reference value, where that patient is specifically chosen from a larger group of patients on the basis of the particular patient having a particular level of expression of epidermal growth factor receptor that is higher or lower than the reference value. By selecting, selectively treating and selectively administering, it is meant that a patient is delivered a personalized therapy based on the patient's particular biology, rather than being delivered a standard treatment regimen based solely on the patient having a particular disease. Thus, selective treatment differs from standard treatment, which delivers a particular drug to all patients, regardless of their particular level of expression of epidermal growth factor receptor.

Numerous quantitative image analysis procedures are known in the art. An example of a quantitative image analysis procedure that may be used to determine the levels of expression include AQUA® technology procedures, as described in issued U.S. Pat. No. 7,219,016, U.S. Pat. No. 8,036,833 and U.S. Pat. No. 8,121,794, which are incorporated by reference in to this application in its entirety.

Other quantitative image analysis procedures may include the Bliss system, the ACIS system, the IVision and GenoMx system, the ScanScope Systems, the Ariol SL-50 System, the Vectra and Nuance systems, Leica microscope systems, and the LSC system which are available from the following respective manufacturers: Bacus Laboratories, Inc., Clarient, Inc., BioGenex, DakoCytomation, Applied Imaging Corporation, Perkin Elmer (Caliper), Leica and CompuCyte Corporation (for more information, please see Immunohistochemistry and Quantitative Analysis of Protein Expression, by Melissa Cregger, Aaron J. Berger, and David L. Rimm, published July 2006 in Archives of Pathology and Laboratory Medicine); the procedure described in The Relative Distribution of Membranous and Cytoplasmic Met is a Prognostic Indicator in Stage I and II Colon Cancer, by Fiora Ginty, Sudeshna Adak, Ali Can, Michael Gerdes, Melinda Larsen, h arvey Cline, Robert Filkins, Zhengyu Pang, Qing Li, and Michael C. Montalto, published Jun. 15, 2008 in Clinical Cancer Research; and the procedure described in Quantitative Fluorescence Imaging Analysis for Cancer Biomarker Discovery: Applications to β-Catenin in Archives Prostate Specimens, by Dali Huang, George P. Casale, Jun Tian, Nizar K. Wehbi, Neil A. Abrahams, Zahid Kaleem, Lynette M. Smith, Sonny L. Johansson, Johny E. Elkahwaji, and George P. Hemstreet III published July 2007 in Cancer Epidemiology Biomarkers). The disclosures of these publications is hereby incorporated by reference into this application.

An assay to measure the level of expression of EGFR and specifically using an antibody having the characteristics of D38B1 has been described in Dimou et al., 179:580-9, 2011, the contents of which is hereby incorporated by reference into this application.

Analytic Variability in Immunohistochemistry Biomarker Studies, Anagnostou, Cancer Epidemiol. Boamarkers Prev.; 19(4), April 2010, the contents of which is hereby incorporated by reference into this application, illustrates how one can regress the results obtained with one antibody with another anti-EGFR antibody, determine their correlation, and thereby select an antibody having a desired specificity for the assay.

Abbreviations that may be used throughout the application include the following:
AQUA: Automated quantitative analysis
CI: Confidence interval
EGF: Epidermal growth factor
EMT: Epithelial to mesenchymal transition
ER: Estrogen receptor
FFPE: Formalin fixed paraffin embedded
HR: Hazard ratio
MQIF: Multiplex quantitative immunofluorescence
PR: Progesterone receptor

### Q: Quartile

TMA: Tissue microarray
TMEM: Tumor microenvironment for metastasis

The present invention provides, among other things, methods for treating a patient having a tumor associated with HER2+ breast cancer. While specific embodiments of the subject invention have been discussed, the specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

Throughout this application, various publications are referenced by footnotes, endnotes, and/or parentheses. The disclosures of each of the publications found in this specification is hereby incorporated by reference into this application in order to more fully describe the state of the art as known to those skilled therein as of the date of this application. The following Experimental Details are set forth to aid in an understanding of the subject matter of this disclosure, but are not intended to, and should not be construed to, limit in any way the claims which follow thereafter.

### Experimental Details

Human Epidermal Growth Factor 1 (EGFR) is a key member of the HER signaling pathway and may be associated with trastuzumab resistance. Cell line models have revealed that over-expression and activation of EGFR induces trastuzumab resistance in HER2 positive cell lines. (Dua at al., Breast Cancer Res Treat 122:685-97, 2010 and Kumar et al., Clin Cancer Res 13:4657-9, 2007). However, data supporting EGFR assessment as a biomarker of trastuzumab resistance in breast cancer patients remains inconclusive. Perhaps the most critical variable preventing common usage of EGFR is non-reproducibility. Many antibodies have been generated for EGFR assessment, but most require a protease treatment in the process of antigen retrieval for immunohistochemistry. The lack of reproducibility associated with this issue and the fact that the agreement between different commercially available EGFR antibodies is poor (Anagnostou at al., Cancer Epidemiol Biomarkers Prev 19:982-91, 2010) and has resulted in a confusing literature on assessment of the impact of EGFR testing and the lack of its adoption as a companion diagnostic test. Recently, a new monoclonal antibody for EGFR (called D38B1) has been developed to the cytoplasmic domain of the molecule that does not require protease treatment for antigen retrieval. Using this antibody, our group has standardized a sensitive, specific and reproducible fluorescence-based, quantitative EGFR assay (Dimou at al., Am J Pathol 179:580-9, 2011). In this study, we used this assay to evaluate the association between EGFR expression and trastuzumab response in the North Central Cancer Treatment Group (NCCTG) N9831 trial. We then validate the result in a retrospectively collected trastuzumab-treated metastatic breast cancer cohort from Greece.

NCCTG N9831 trial assessed the efficacy and safety of adding 52 weeks of trastuzumab to standard anthracycline/taxane-based chemotherapy (doxorubicin plus cyclophosphamide [AC] followed by paclitaxel) arm. It enrolled a total of 3505 patients, randomly assigned to trastuzumab contained chemotherapy regime and control chemotherapy treatment regime. The trial was among the four large clinical trials that led to FDA approval of the usage of trastuzumab in adjuvant setting. In this study, a tissue microarray (TMA) containing 1580 samples in three fold redundancy from this trial was assessed for EGFR expression using the recently described, standardized EGFR assay. Our goal was to determine if EGFR expression was associated with benefit from trastuzumab in the adjuvant setting.

### Methods and Materials

### Cohort Description

The NCCTG N9831 trial is a three-arm prospective phase III randomized trial. Eligible patients were randomly assigned into three arms. Arm A is a control arm, in which patients received AC followed by weekly paclitaxel; Arm B is a sequential arm, in which patients received AC followed by weekly paclitaxel followed by trastuzumab; and Arm C is a concurrent arm, in which patients received AC followed by weekly paclitaxel plus trastuzumab followed by trastuzumab alone. Radiation and/or hormonal therapy were administered after completion of chemotherapy, when indicated. Patients were enrolled as a collaborative effort between NCCTG and other NCI-sponsored cancer cooperative groups (ECOG, SWOG, CALGB).

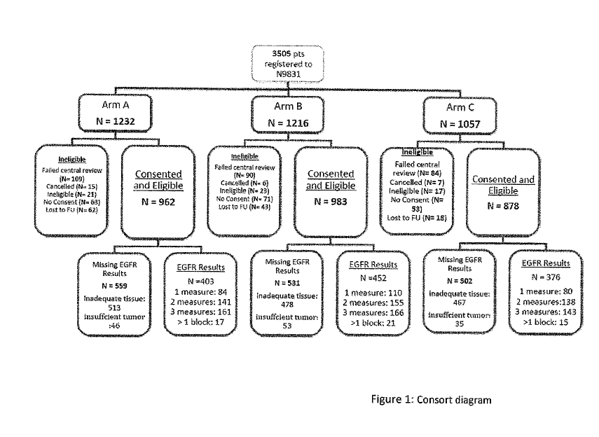

A total of 3505 women were registered to N9831 of which 2823 women provided consent and were deemed eligible upon central review. For this study, there were 1231 women in the tissue microarray (TMA) for evaluation (403 from Arm A, 452 from Arm B, 376 from Arm C; see FIG. 1) and 1458 women not present in the TMA: 1458 due to inadequate tissue (513 Arm A, 478 Arm B, 467 Arm C) and 134 due to tissue loss in the TMA-based assays (46 Arm A, 53 Arm B, and 35 Arm C). Of the 1231 women with measurements, 274 women had one measurement, 434 had 2 measurements, 470 women had 3 measurements, and 53 women had measurements from more than one block. Average scores were used to represent each patient.

The second cohort as a validation cohort is a retrospectively collected metastatic breast cancer cohort treated with various chemotherapy regimens with trastuzumab was obtained collaboratively from Dr. Amanda Psyrri in Greece. The TMAs contain 149 patients up to four-fold redundancy. Of the 149 patients, 130 patients received trastuzumab in first-line treatment and were included in the analysis. In these 130 patients, four have four measurements, four have three measurements, 65 have two measurements, and 35 have one measurement. Average scores were used to represent each patient. The detailed cohort descriptions are shown in Table 1.

TABLE 1A
Patient, tumor and disease characteristics for patients with and
without EGFR measurements for women in the N9831 cohort . . .

| | Patients in the | | Patients not | | |
|---|---|---|---|---|---|
| | TMA | | in the TMA | | |
| Characteristic | N = 1231 | | N = 2274 | | p-value |
| Arm assignment, n (%) | | | | | |
| A | 403 | (33%) | 829 | (36%) | |
| B | 452 | (37%) | 764 | (34%) | |
| C | 376 | (31%) | 681 | (30%) | 0.06 |
| Age at randomization, n (%) | | | | | |
| 18-39 years | 222 | (18%) | 365 | (16%) | |
| 40-49 years | 390 | (32%) | 768 | (34%) | |
| 50-59 years | 392 | (32%) | 749 | (33%) | |
| ≧60 years | 227 | (18%) | 392 | (17%) | 0.07 |
| Extent of surgery, n (%) | | | | | |
| Breast sparing | 451 | (37%) | 906 | (40%) | |
| Mastectomy | 780 | (63%) | 1368 | (60%) | 0.06 |
| Extent of nodal | | | | | |
| examination, n (%) | | | | | |
| Axillary node dissection | 611 | (50%) | 989 | (43%) | |
| Sentinel biopsy | 620 | (50%) | 1285 | (57%) | 0.0005 |
| Tumor size, n (%) | | | | | |
| ≦2.0 cm | 452 | (37%) | 941 | (41%) | |
| 2.1-4.9 cm | 624 | (51%) | 1104 | (49%) | |
| ≧5.0 cm | 155 | (13%) | 229 | (10%) | 0.008 |
| Histologically positive | | | | | |
| nodes, n (%) | | | | | |
| 0 | 143 | (12%) | 328 | (14%) | |
| 1-3 | 502 | (41%) | 900 | (40%) | |
| 4-9 | 173 | (14%) | 278 | (12%) | |
| ≧10 | 331 | (27%) | 578 | (25%) | |
| Sentinel Node Positive | 82 | (7%) | 190 | (8%) | 0.03 |
| Tumor grade, n(%) | | | | | |
| 1 | 25 | (2%) | 55 | (2%) | |
| 2 | 314 | (26%) | 616 | (27%) | |
| 3 | 882 | (72%) | 1558 | (69%) | |
| Unknown | 10 | (<1%) | 55 | (2%) | 0.32 |
| Estrogen receptor status, | | | | | |
| n (%) | | | | | |
| positive | 603 | (49%) | 1841 | (53%) | |
| negative | 628 | (51%) | 1663 | (47%) | 0.006 |

TABLE 1B
Description of Greek cohort.

| Variables | Number | % |
|---|---|---|
| Age | | |
| <50 | 39 | 26% |
| >50 | 110 | 74% |
| Grade | | |
| 1 | 3 | 2% |
| 2 | 53 | 36% |
| 3 | 83 | 56% |
| Unknown | 10 | 7% |
| Distant Metastasis | | |
| Yes | 132 | 89% |
| No | 11 | 7% |
| Unknown | 6 | 4% |
| ER | | |
| Positive | 104 | 70% |
| Negative | 45 | 30% |
| PR | | |
| Positive | 76 | 51% |
| Negative | 73 | 49% |
| HER2 | | |
| Positive | 90 | 60% |
| Negative | 59 | 40% |
| Trastuzumab | | |
| 1st Line | 130 | |
| Monotherapy | 4 | 3% |
| with Anthracycline | 24 | 18% |
| with Taxane | 100 | 77% |
| 2nd Line + | 19 | |
| Monotherapy | 3 | 16% |
| with Anthracycline | 4 | 21% |
| with Taxane | 7 | 37% |

### Quantitative Immunofluorescence Based Assessment of EGFR

TMA slides were prepared at Mayo Clinic and shipped to Yale (DR's lab) where they were deparaffinized in xylene and rehydrated with alcohol. Antigen retrieval was performed using a PT module (LabVision Corp., Fremont, Calif.) with EDTA buffer, pH 8, at 97° C. for 20 minutes. Slides are then incubated in 2.5% hydrogen peroxide for 30 minutes at room temperature to block endogenous peroxidase activity. The following steps were then done in an autostainer (LabVision Corp., Fremont, Calif.). Nonspecific antigens were blocked via incubation in 0.3% bovine serum albumin in Tris-buffered saline solution and Tween 20 for 30 minutes at room temperature. Slides were then incubated overnight with a cocktail of a primary EGFR antibody clone D38B1 (Cell Signaling Technology, Danvers, Mass.) used at 1:200 titter, and a mouse monoclonal cytokeratin antibody (Dako Corp., Carpinteria, Calif.). Next, a cocktail of Alexa 546-conjugated goat anti-mouse secondary antibody (Molecular Probes, Inc., Eugene, Oreg.) diluted 1:100 in rabbit Envision reagent (Dako Corp.) was applied to the slides for 1 hour at room temperature. To develop fluorescent signal, cyanine 5-tyramide (PerkinElmer, Inc., Waltham, Mass.) diluted 1:50 was used at room temperature for 10 minutes. Finally, Prolong Gold (Molecular Probes, Inc.) containing DAPI was used to detect nuclei. An index array was stained aside each cohort array to enable standardization of the assay, along with negative (no primary) and positive controls.

AQUA method of quantitative immunofluorescence (QIF) was used to quantitatively measure EGFR expression. The method used was exactly that described recently for EGFR in lung cancer (Dimou et al., Am J Pathol 179:580-9, 2011 and in other reviews (Camp at al., Nat Med 8:1323-7, 2002 and Gustayson et al., Appl Immunohistochem Mol Morphol 17:329-37, 2009). In brief, monochromatic fluorescent images of nuclei (DAPI channel), cytokeratin (cy3 channel) and target EGFR (cy5 channel) were captured using a microscope (PM-2000; HistoRx, Inc., Branford, Conn.). A binarized tumor mask and subcellular compartment were generated by the DAPI image and the cytokeratin image using the clustering algorithm in AQUA analysis software. The AQUA scores of EGFR in the tumor mask were calculated as the sum of the target EGFR intensity in the tumor mask divided by the area of the tumor mask. The AQUA scores were normalized for exposure time, bit depth, and lamp hours for optimal standardization and reproducibility.

In order to calculate the absolute EGFR level in the cell, two steps of normalization were applied. The AQUA score of EGFR measured in N9831 cohort was first normalized to a standardized index array, which consists of a series of lung cancer cases and cell lines. This index array is the same as previously used to define EGFR concentrations in a lung cancer study⁶. Then the normalized AQUA score was translated into concentration units (nanograms of EGFR per microgram of total protein) of EGFR using the standard curve was derived from the previous study⁶.

### Statistical Analysis

Statistical calculations were performed using R and StatView (SAS Cary, N.C.). Disease specific survival was used as the endpoint in the NCCTG N9831 cohort analysis. Continuous scores were applied in the cox proportional hazard survival model to test the correlation with survival outcome. Optimal cutoff point of continuous EGFR was generated by R-part in Arm C in NTTCG N9831 cohort (EGFR level=13 ng/μg). The same cutoff point was applied to Arm A and Arm B, survival difference were tested using Kaplan-Meier estimates. Stratification factors were used in the multivariate cox proportional hazard model. The Greek cohort was used as a validation cohort, and progression free survival was chosen as the endpoint for the predictive value assessment in a metastatic setting. All the statistical tests were performed two-sided at a significance level of p=0.05.

### Results:

A comparison of the 1231 women with an EGFR measurement to the 2274 without an EGFR measurement revealed some statistically significant differences between the groups (Table 1). On average, women with EGFR measures were more likely to have an axillary node dissection, had larger tumors, had more lymph node involvement and were less likely to be estrogen receptor negative. Many of these differences reflect the fact that women with more disease were more likely to have greater amounts of tissue available for TMA production and correlative studies.

High Expression of EGFR is Associated with Worse Survival Outcome in NCCTG N9831 Cohort

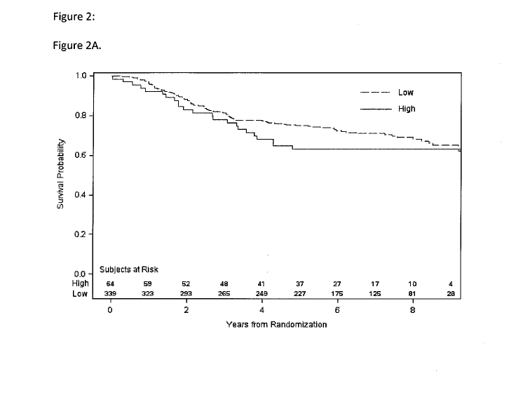

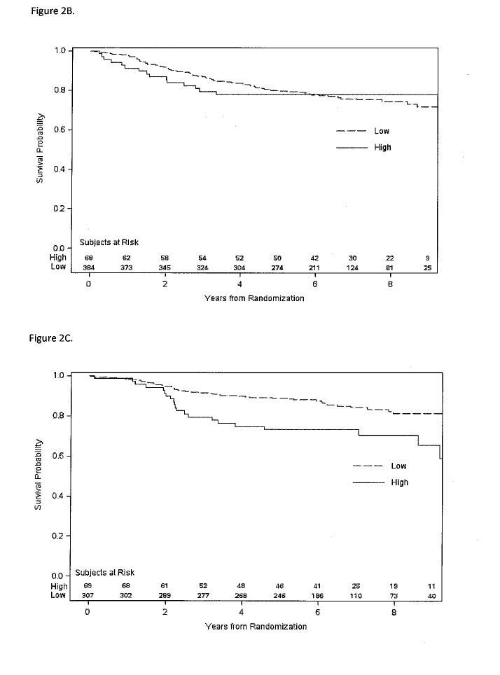

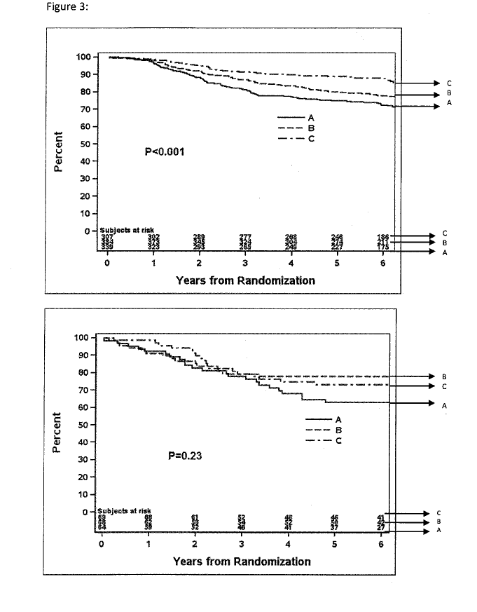

The cutpoint determined by rpart was 840 (or EGFR level of 13 ng/μg), which also corresponds to the median value of women with elevated levels of EGFR. Women with an EGFR AQUA score above 840 were classified as the EGFR high group; there 201 out of 1231 women in this group (16.3%): 64 (15.9%) in Arm A, 68 (15.0%) in Arm B, and 69 (18.4%) in Arm C. The survival curves in FIG. 2 shows that women with high EGFR in Arm C have a significantly poorer DFS outcome (FIG. 2C): the percent of women who are disease free at 5-years is 88.8 compared to 73.2% for women with low EGFR in Arm C (p-value=0.003). Although women with high EGFR in Arms A and B have slightly worse DFS, the differences were not statistically significant (FIGS. 2A and 2B). Survival curves were also constructed to compare the arms of the trial within the low (FIG. 3A) and high (FIG. 3B) EGFR groups. Patients with low EGFR show stratification by treatment arm as seen in the original trial. Patients with high EGFR show no benefit in the trastuzumab arms compared to the no trastuzumab arm.

The association between EGFR status (high versus not high) and DFS was evaluated with univariable and multivariable models (Table 2) for each of the three treatment arms. The association was statistically significant for Arm C in both the univariable and multivariable models, but not for Arms A and B in either model. Multivariate analysis of all collected clinical variables within arm C shows that EGFR is an independent predictive variable (table 3). A test for interaction between EGFR status and the treatment arms (Arm A versus Arm C) did not achieve statistical significance in both the univariable model (p-value=0.15) and the multivariable model (p-value=0.15), which could be due to limited power to detect an interaction.

TABLE 2
Cox proportional hazard models evaluating the association
between EGFR group and DFS.

| | univariable model | | multivariable model | |
|---|---|---|---|---|
| | HR (95% CI) | p-value | HR (95% CI) | p-value |
| Arm A | 1.29 (0.82-2.04) | 0.26 | 1.18 (0.74-1.89) | 0.49 |
| Arm B | 0.95 (0.55-1.63) | 0.84 | 0.91 (0.52-1.61) | 0.75 |
| Arm C | 2.15 (1.28-3.60) | 0.004 | 1.83 (1.05-3.17) | 0.03 |

TABLE 3
Multivariate Cox Proportional Hazard Model (Time to Progression):
Arm C Only

| | | 95% Confidence | | |
|---|---|---|---|---|
| Variable | Hazard Ratio | Interval | Chi-Sq | p |
| Age Group | | | | |
| >50 vs <50 | 1.57 | 0.952-1.580 | 3.122 | 0.077 |
| Grade | | | | |
| III vs I and II | 1.77 | 0.950-3.283 | 3.233 | 0.072 |
| ER | | | | |
| Positive vs Negative | 0.76 | 0.439-1.327 | 0.917 | 0.338 |
| Nodal Status | | | | |
| Negative vs Positive | 0.72 | 0.334-1.554 | 0.699 | 0.403 |
| EGFR | | | | |
| High vs Low | 1.83 | 1.048-3.176 | 4.523 | 0.033 |

### Validation in the Greek Cohort

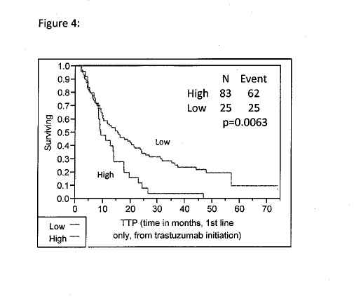

Although the univariate analysis reveals a continuous relationship between EGFR and DFS in Arm C, the data were dichotomized to classify patients for future studies as positive or negative. Since there is not prescribed cut-point in the continuous data, a regression tree method was used to define the value of 13 ng/ug. Although this is validated by significance in Arm C, but not Arms A and B, the validity is strengthened by secondary validation on an independent cohort. Although an adjuvant cohort would be more similar, for practical purposes and to increase the biological robustness of the test, a metastatic cohort was selected from a small multi-institutional trial in Greece. The cohort contains 130 patients treated with first line trastuzumab. Continuous assessment of EGFR using the Cox proportional hazard model with progression free survival as the end point, was statistically significant (p=0.0366). When EGFR was dichotomized at 13 ng/μg, and assessed by KM analysis, the median difference in progression free survival was 6.1 months (log rank p=0.0063) (FIG. 4). The univariate binary HR is 1.92 (95% CI: 1.192-3.092, p=0.0073). The association between EGFR and PFS remained significant when age, grade, distant metastasis, ER IHC and HER2 status were controlled (Table 4).

TABLE 4
Multivariate Cox Proportional Hazard Model in the Greek cohort
Multivariate Cox Proportional Hazard Model (Time to Progression)

| | | 95% Confidence | | |
|---|---|---|---|---|
| Variable | Risk Ratio | Interval | Chi-Sq | p |
| Age Group | | | | |
| >50 vs <50 | 0.797 | 0.492-1.338 | 0.769 | 0.3805 |
| Grade | | | | |
| III vs I and II | 1.022 | 0.644-1.639 | 0.008 | 0.9274 |
| ER | | | | |
| Positive vs Negative | 0.701 | 0.399-1.235 | 1.518 | 0.2179 |
| HER2 | | | | |
| Positive vs Negative | 0.609 | 0.357-1.048 | 3.216 | 0.0729 |
| HER1 | | | | |
| High vs Low | 1.790 | 1.033-3.036 | 4.301 | 0.0381 |

Thus, EGFR assessed as a continuous variable shows an association with disease free survival (DFS) in Arm C (p=0.0016) but not in Arm A or B. Dichotimizing EGFR expression shows that high level expression is associated with worse outcome (HR=2.2; 95% CI 1.33-3.59, p=0.002). A Kaplan Meir plot shows 5 year DFS within the low level EGFR expression group as 71%, 74% and 90% for Arms A, B and C, respectively (log-rank p-value=0.02) and within the high level EGFR expression group as 62%, 76% and 74% for Arms A, B and C, respectively (log-rank p-value 0.22). As validation, the same cut-point was used define high EGFR expression in a retrospective metastatic breast cancer cohort. The median PFS difference between EGFR high group and EGFR low group was 6.1 months (p=0.0063) and the hazard ratio of high EGFR was 1.92 (p=0.0073).

High expression of EGFR is associated with decreased benefit from adjuvant concurrent (not sequential) trastuzumab and shorter PFS in the metastatic setting. Since there are other treatment options for HER2-driven tumors, further validation of our data may select patients for alternative or additive therapy.

### DISCUSSION

Although EGFR expression has been associated with trastuzumab resistance in cell line models, previous biomarker studies of EGFR have not validated this observation in human tumors. Initially an EGFR assay was established as a companion diagnostic assay with early trials of cetuximab in colon cancer. Ultimately, studies showed that EGFR status by IHC made no difference in outcome (Lenz et al., J Clin Oncol 24:4914-21, 2006) and CAP surveys of labs were unable to reach consensus, so the assay was largely discarded. Here we used a standardized antibody specific for the cytoplasmic domain of EGFR (D38B1) without a protease-based antigen retrieval to determine EGFR expression, and furthermore to exact protein concentrations (Dimou et al., supra.)

An interesting and somewhat unexpected result seen in our analysis is that high EGFR was associated with lack of benefit from trastuzumab in the 393 patients concurrent trastuzumab treatment arm but not seen when trastuzumab was given in sequence rather than concurrently.

The QIF method used here generates data on a continuous scale, but treatment decisions are binary. Thus one approach for use as a companion diagnostic test, is to dichotomize the continuous variables to better direct therapy. Since there is no known biological cut-point indicating the level at which EGFR shows its effect, we used the R-part algorithm to attempt to discover the most meaningful cut-point for use in future studies. We were able to test a retrospective metastatic cohort which validated the discovered cut-point.

The observation is compelling because of the mechanistic relationship between EGFR and HER2 and because of the availability of the dual inhibitor lapatinib. While the ALTTO trial is still underway, the Neo-ALTTO trial showed benefit in both the trastuzumab and lapatinib arms with the maximal benefit seen in the combination arm. Our study suggests that EGFR expression measured on the patients in that trial could identify the subset of patients with high EGFR expression may show better response in the lapatinib or combination arms compared to the arm of those treated with trastuzumab alone.

### REFERENCES

1. Perez E A, Romond E H, Suman V J, et al: Four-year follow-up of trastuzumab plus adjuvant chemotherapy for operable human epidermal growth factor receptor 2-positive breast cancer: joint analysis of data from NCCTG N9831 and NSABP B-31. J Clin Oncol 29:3366-73, 2011
2. Zito C R, Jilaveanu L B, Anagnostou V, et al: Multi-level targeting of the phosphatidylinositol-3-kinase pathway in non-small cell lung cancer cells. PLoS One 7:e31331, 2012
3. Dua R, Zhang J, Nhonthachit P, et al: EGFR over-expression and activation in high HER2, ER negative breast cancer cell line induces trastuzumab resistance. Breast Cancer Res Treat 122:685-97, 2010
4. Kumar R: ErbB-dependent signaling as a determinant of trastuzumab resistance. Clin Cancer Res 13:4657-9, 2007
5. Anagnostou V K, Welsh A W, Giltnane J M, et al: Analytic variability in immunohistochemistry biomarker studies. Cancer Epidemiol Biomarkers Prev 19:982-91, 2010
6. Dimou A, Agarwal S, Anagnostou V, at al: Standardization of Epidermal Growth Factor Receptor (EGFR) Measurement by Quantitative Immunofluorescence and Impact on Antibody-Based Mutation Detection in Non-Small Cell Lung Cancer. Am J Pathol 179:580-9, 2011
7. Camp R L, Chung G G, Rimm D L: Automated subcellular localization and quantification of protein expression in tissue microarrays. Nat Med 8:1323-7, 2002
8. Gustayson M D, Bourke-Martin B, Reilly D M, et al: Development of an unsupervised pixel-based clustering algorithm for compartmentalization of immunohistochemical expression using Automated Quantitative Analysis. Appl Immunohistochem Mol Morphol 17:329-37, 2009
9. Lenz H J, Van Cutsem E, Khambata-Ford S, et al: Multicenter phase II and translational study of cetuximab in metastatic colorectal carcinoma refractory to irinotecan, oxaliplatin, and fluoropyrimidines. J Clin Oncol 24:4914-21, 2006
10. Xia L, Wang L, Chung A S, et al: Identification of both positive and negative domains within the epidermal growth factor receptor COOH-terminal region for signal transducer and activator of transcription (STAT) activation. J Biol Chem 277:30716-23, 2002
11. Scaltriti M, Verma C, Guzman M, et al: Lapatinib, a HER2 tyrosine kinase inhibitor, induces stabilization and accumulation of HER2 and potentiates trastuzumab-dependent cell cytotoxicity. Oncogene 28:803-14, 2009

1. A method for treating a patient having a tumor associated with HER2+ breast cancer comprising:
a) measuring in a sample of the patient's tumor a level of expression of epidermal growth factor receptor protein by using an antibody that detects the cytoplasmic domain of epidermal growth factor receptor; and
b) comparing the level of expression of epidermal growth factor receptor protein in the patient's tumor so measured with a reference value;
wherein if the level of expression of epidermal growth factor receptor measured in step a) is higher than the reference value, treating the patient with a therapy other than trastuzumab.

2. The method of claim 1, wherein if the level of expression of epidermal growth factor receptor measured in step b) is higher than the reference value, treating the patient with a therapy other than chemotherapy involving concurrent administration of trastuzumab.

3. The method of claim 1, wherein the level of expression is determined using a quantitative image analysis procedure.

4. The method of claim 1, wherein the level of expression is determined using an automated pathology system.

5. The method of claim 1, wherein the reference value is thirteen nanograms of epidermal growth factor receptor protein per microgram of total protein.

6. The method of claim 1, wherein the antibody detects the epidermal growth factor receptor epitope that is bound by D38B1.

7. The method of claim 1, wherein the antibody has the specificity of D38B1.

8. (canceled)

9. The method of claim 1, wherein the level of expression of epidermal growth factor receptor protein is the level within a subcellular compartment.

10. (canceled)

11. (canceled)

12. (canceled)

13. A method for treating a patient having a tumor associated with HER2+ breast cancer comprising:
a) measuring in a sample of the patient's tumor a level of expression of epidermal growth factor receptor protein by using an antibody that detects the cytoplasmic domain of epidermal growth factor receptor;
b) comparing the level of expression of epidermal growth factor receptor protein in the patient's tumor so measured with a reference value; and
c) treating the patient with a therapy other than chemotherapy involving concurrent administration of trastuzumab
if the level of expression of epidermal growth factor receptor measured in step a) is higher than the reference value.

14. The method of claim 13, wherein the level of expression is determined using a quantitative image analysis procedure.

15. The method of claim 13, wherein the level of expression is determined using an automated pathology system.

16. The method of claim 13, wherein the reference value is thirteen nanograms of epidermal growth factor receptor protein per microgram of total protein.

17. The method of claim 13, wherein the antibody detects the epidermal growth factor receptor epitope that is bound by D38B1.

18. The method of any of claim 13, wherein the antibody has the specificity of D38B1.

19-115. (canceled)

116. A method for determining the likelihood that a HER2+ breast cancer patient will benefit from trastuzumab comprising measuring the level of expression of epidermal growth factor receptor in a tumor specimen obtained from the patient comprising:
a) staining a tissue sample of the tumor with a first stain that labels a subcellular compartment wherein the subcellular compartment is a non-nuclear compartment and a second stain that labels a biomarker, wherein the biomarker is epidermal growth factor receptor protein and wherein the second stain is specific for the cytoplasmic domain of epidermal growth factor receptor;
b) obtaining a high resolution digital image of each of the epidermal growth factor receptor and of the subcellular compartment;
c) analyzing the digital images to determine a quantitative level of expression of epidermal growth factor receptor protein present in the subcellular compartment;
d) comparing the level of expression of epidermal growth factor receptor protein so determined with a reference value;
thereby determining the level of expression of epidermal growth factor receptor protein in the tumor, wherein if the level of expression of epidermal growth factor receptor is higher than the reference value, it is unlikely the patient will benefit from trastuzumab.

117. The method of claim 116, wherein if the level of expression of epidermal growth factor receptor is higher than the reference value, treating the patient with a therapy other than chemotherapy administered with concurrent trastuzumab.

118. The method of claim 116, wherein the tissue section is fixed.

119. The method of claim 116, wherein the antibody detects the epidermal growth factor receptor epitope that is bound by D38B1.

120. The method of claim 116, wherein the antibody has the specificity of D38B1.

121. The method of claim 116, wherein the reference value is thirteen nanograms of epidermal growth factor receptor protein per microgram of total protein.

122. (canceled)
